(19) United States
(12) Patent Application Publication
(10) Pub. No.: US 2024/0339020 A1
(43) Pub. Date: Oct. 10, 2024

(54) Health Monitoring Assembly

(71) Applicant: Tanquenica Strahan, Dallas, TX (US)

(72) Inventor: Tanquenica Strahan, Dallas, TX (US)

(21) Appl. No.: 18/131,578

(22) Filed: Apr. 6, 2023

(51) Int. Cl. G08B 21/04; A61B 5/00; G04G 21/02; G04G 21/04; G08B 3/10; G08B 5/22; G08B 5/36; G08B 7/06; G08B 25/10

(52) U.S. Cl. CPC G08B 21/0453; A61B 5/681; A61B 5/746; A61B 5/747; G04G 21/025; G04G 21/04; G08B 3/1016; G08B 5/228; G08B 5/36; G08B 7/06; G08B 21/0476; G08B 25/10; A61B 5/748

(57) ABSTRACT

A health monitoring assembly includes a wrist watch. Each of a pair of engagement elements is integrated into the wrist watch. A communication unit is integrated into the wrist watch and the communication unit is in remote communication with emergency responders. A plurality of biometric sensors is each integrated into the wrist watch thereby facilitating each of the biometric sensors to be in communication with the user's body to sense a respective one of a plurality of biometric parameters of the user's body. Each of the biometric sensors is in communication with the communication unit and the communication unit communicates a distress alert to the emergency responders when any of the plurality of biometric sensors senses that any of the respective biometric parameters of the user's body is outside of a pre-determined range. In this way the plurality of biometric sensors facilitate the user to request emergency assistance.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to monitoring devices and more particularly pertains to a new monitoring device for monitoring the health of an elderly person and contacting emergency responders when the health of the elderly person is compromised. The device includes a wrist watch and a plurality of biometric sensors that are each integrated into the wrist watch. The device includes a communication unit that is in wireless communication with an extrinsic communication network for contacting the emergency responders.

### (2) Description of Related Art Including Information Disclosed Under 37 CFR 1.97 and 1.98

The prior art relates to monitoring devices including a smart watch which includes a touch screen that displays an interactive image. The prior art discloses a smart watch with video call functionality. The prior art discloses a vital sign monitoring watch that includes a device receiving portion and a communication port. The prior art discloses a life alert system which monitors a subjects heart rate and which alerts emergency responders in the event of a cardiac incident. The prior art discloses a blood pressure measuring device that includes an annular bracelet, a display integrated into the annular bracelet, a camera disposed on the annular bracelet and a blood pressure monitor. The prior art discloses a wearable security device that includes a wrist strap with a heart rate monitor and a wireless alert. The prior art discloses a system and method for predicting an unnecessary emergency room visit based on data collected by an activity tracker on a smart watch.

### BRIEF SUMMARY OF THE INVENTION

An embodiment of the disclosure meets the needs presented above by generally comprising a wrist watch that is positionable on a user's wrist. A pair of engagement elements is each integrated into the wrist watch, a communication unit is integrated into the wrist watch and the communication unit is in remote communication with emergency responders. A plurality of biometric sensors is each integrated into the wrist watch thereby facilitating each of the biometric sensors to be in communication with the user's body to sense a respective one of a plurality of biometric parameters of the user's body. Each of the biometric sensors is in communication with the communication unit and the communication unit communicates a distress alert to the emergency responders when any of the plurality of biometric sensors senses that any of the respective biometric parameters of the user's body is outside of a pre-determined range. In this way the plurality of biometric sensors facilitate the user to request emergency assistance.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a health monitoring assembly according to an embodiment of the disclosure.

FIG. 2 is a top view of a wrist watch of an embodiment of the disclosure.

FIG. 3 is a bottom view of a wrist watch of an embodiment of the disclosure.

FIG. 4 is a cross sectional view taken along line 4-4 of FIG. 3 of an embodiment of the disclosure showing a sliding engagement in a first position.

FIG. 5 is a cross sectional view taken along line 4-4 of FIG. 3 of an embodiment of the disclosure showing a sliding engagement in a second position.

FIG. 6 is a perspective in-use view of an embodiment of the disclosure.

FIG. 7 is a schematic view of an embodiment of the disclosure.

### DETAILED DESCRIPTION OF THE INVENTION

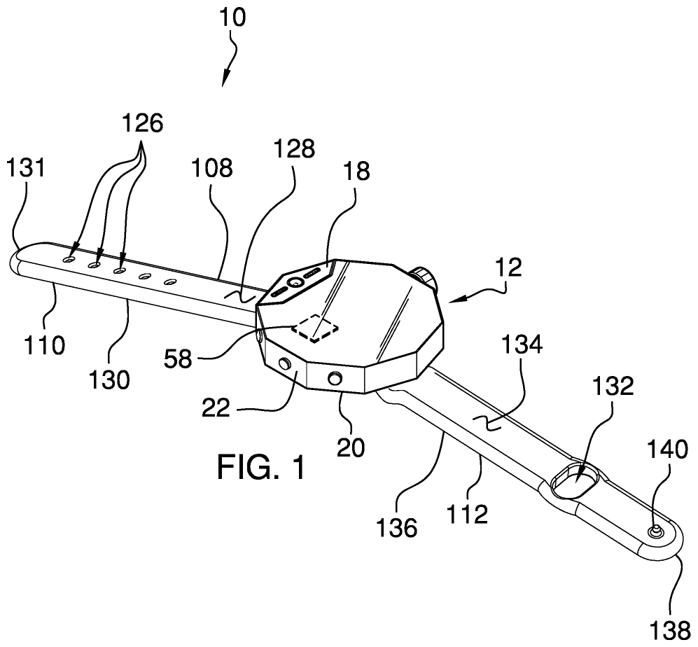

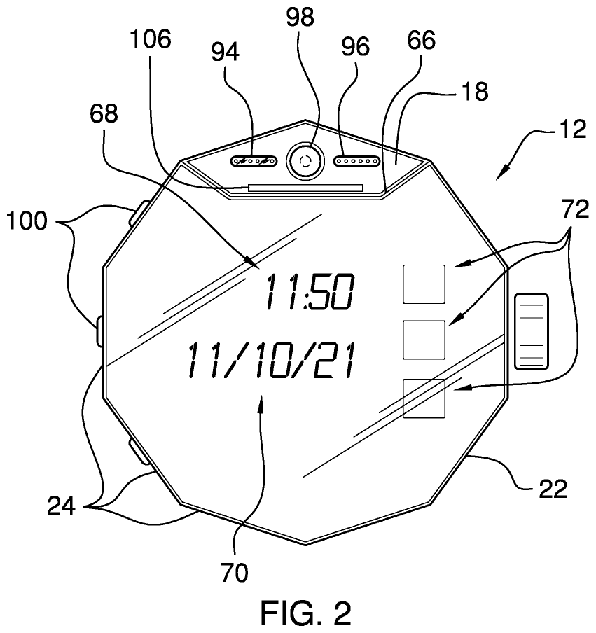

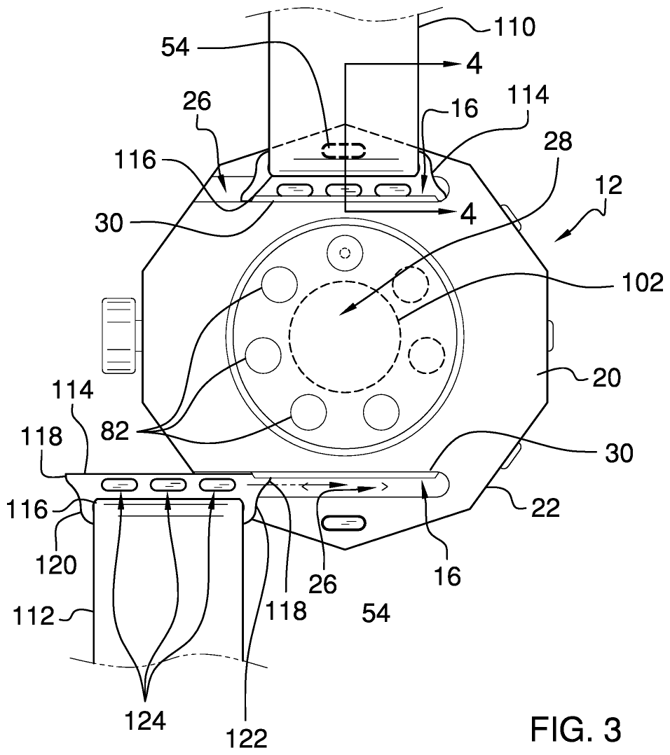

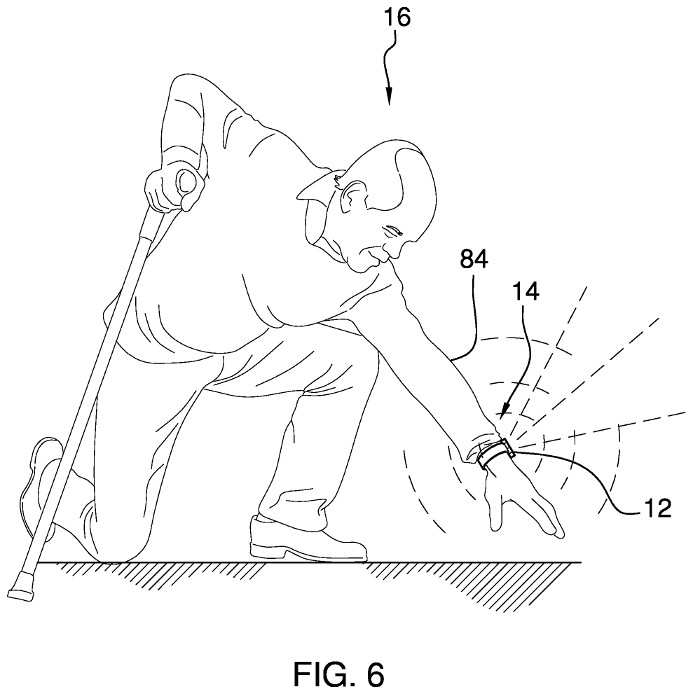

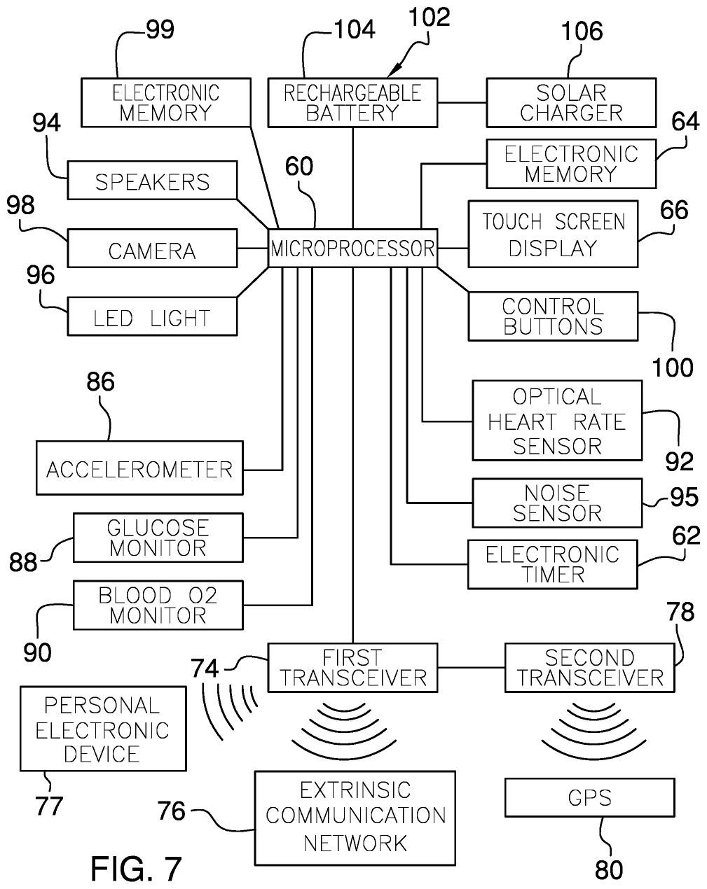

With reference now to the drawings, and in particular to FIGS. 1 through 7 thereof, a new monitoring device embodying the principles and concepts of an embodiment of the disclosure and generally designated by the reference numeral 10 will be described.

As best illustrated in FIGS. 1 through 7, the health monitoring assembly 10 generally comprises a wrist watch 12 that is positionable on a user's wrist 14 and the wrist watch 12 has a pair of engagement elements 16 that is each integrated into the wrist watch 12. The user 16 may be an elderly person that is hearing impaired or other physically limited user 16 that is prone to experiencing health problems and who would typically have difficulty contacting emergency responders in the event of a health emergency. The wrist watch 12 has a top wall 18, a bottom wall 20 and a perimeter wall 22 extending between the top wall 18 and the bottom wall 20 and the perimeter wall 22 has a plurality of intersecting sides 24 such that the wrist watch 12 has a decagonal shape. The wrist watch 12 has a pair of slots 26 each extends into the bottom wall 20 such that each of the slots 26 is associated with a respective one of the engagement elements 16 and each of the slots 26 extending laterally across a substantial width of the bottom wall 20. Each of the slots 26 intersects a respective one of the plurality of intersecting sides 24 of the perimeter wall 22. Each of the slots 26 is oriented parallel with each other and the pair of slots 26 is spaced apart from a center 28 of the bottom wall 20.

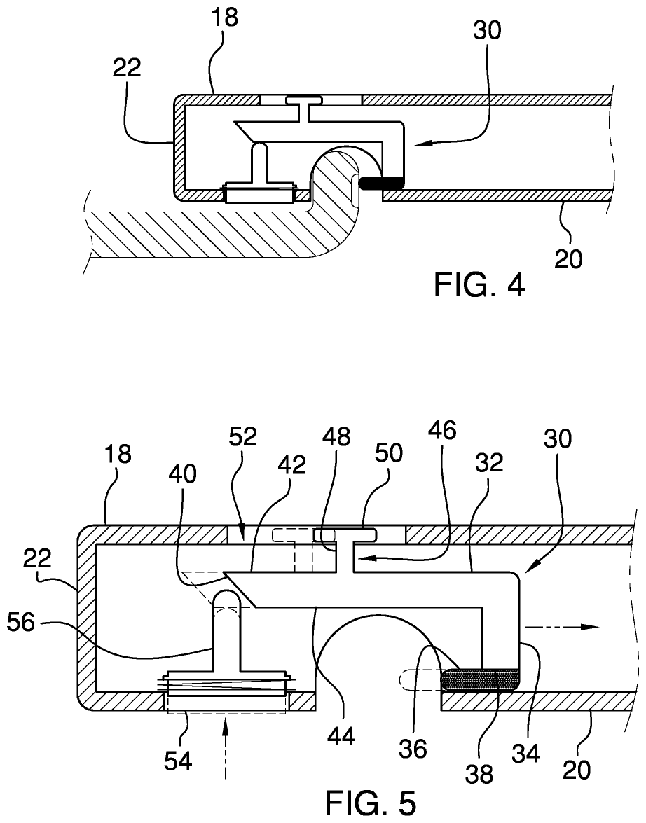

A pair of sliding engagements 30 is each slidably integrated within the wrist watch 12 such that each of the sliding engagements 30 is associated with a respective one of the engagement elements 16. As is most clearly shown in FIGS. 4 and 5, each of the sliding engagements 30 includes a first member 32, a second member 34 oriented at an angle with the first member 32 and a magnet 36 that is disposed on a distal end 38 of the second member 34 with respect to the first member 32. The first member 32 has a front end 40 and the front end 40 is angled such that the front end 40 slopes rearwardly between a top surface 42 of the first member 32 and a bottom surface 44 of the first member 32. The second member 34 is disposed on the bottom surface 44, the second member 34 is perpendicularly oriented with the first member 32 and the magnet 36 is oriented to extend toward the front end 40 of the first member 32. Each of the sliding engagements 30 includes a slide 46 which comprises a stem 48 extending upwardly from the top surface 42 of the first member 32 and a head 50 that is transversely oriented with the stem 48. Furthermore, the head 50 slidably engages a channel 52 that is integrated into the top wall 18 of the wrist watch 12.

Each of the sliding engagements 30 is aligned with a respective one of the slots 26 in the bottom wall 20 of the wrist watch 12. Each of the sliding engagements 30 is biased into a first position having the magnet 36 extending across the respective slot 26. Conversely, each of the sliding engagements 30 is urgeable into a second position having the magnet 36 being displaced from the respective slot 26. Each of the sliding engagements 30 may include a biasing member positioned in the channel 52 in the top wall 18 of the wrist watch 12 which engages the head 50 associated with a respective sliding engagement 30.

A pair of release buttons 54 is each movably integrated into the bottom wall 20 of the wrist watch 12. Each of the release buttons 54 includes a knob 56 which extends into an interior of the wrist watch 12. The knob 56 associated with each of the release buttons 54 is aligned with the front end 40 of the first member 32 of a respective one of the sliding engagements 30. Additionally, the knob 56 of a respective one of the release buttons 54 engages the front end 40 of the first member 32 of the respective sliding engagement 30 when the respective release button 54 is depressed thereby urging the respective sliding engagement 30 into the second position.

A communication unit 58 is integrated into the wrist watch 12 and the communication unit 58 is in remote communication with emergency responders and with emergency contacts of the user such as family members for example. The communication unit 58 comprises a control circuit 60 that is integrated into the wrist watch 12. The control circuit 60 receives an alert input and the control circuit 60 includes an electronic timer 62 for tracking the time of day and the calendar date. Additionally, the control circuit 60 includes an electronic memory 64. A touch screen 66 is integrated into the top wall 18 of the wrist watch 12 thereby facilitating the touch screen 66 to be touched by the user 16. The touch screen 66 is electrically coupled to the control circuit 60 and the touch screen 66 displays time indicia 68 comprising numbers to indicate a time of day. Additionally, the touch screen 66 displays date indicia 70 comprising words and numbers to indicate the calendar date. The touch screen 66 displays menu indicia 72 comprising a graphical user interface thereby facilitating the user 16 to navigate functional parameters of the control circuit 60. The touch screen 66 may comprise a light emitting diode display or other type of electronic display that is touch sensitive.

The communication unit 58 includes a first transceiver 74 that is integrated into the wrist watch 12 and the first transceiver 74 is electrically coupled to the control circuit 60. The first transceiver 74 is in wireless communication with an extrinsic communication network 76 thereby facilitating the first transceiver 74 to be in remote communication with the emergency responders and with the user's 16 emergency contacts. Additionally, the first transceiver 74 is actuated to broadcast a distress alert to the emergency responders when the control circuit 60 receives the alert input as well as to put the user's 16 emergency contacts in communication with the emergency responders. In this way the emergency responders can communicate with the user's emergency contacts to ensure the emergency responders are given pertinent information about the user 16. The first transceiver 74 may comprise a radio frequency transceiver or the like and the extrinsic communication network 76 may comprise the internet, a cellular phone network or any other type of wireless communication network. Furthermore, the first transceiver 74 might be synchronized with the user's personal electronic device 77, such as a smart phone for example, to actuate the smart phone to call the user's 16 emergency contacts in order to alert the user's 16 emergency contacts that the user 16 is experiencing a medical emergency. In this way the user's 16 emergency contacts can immediately respond to the user's 16 emergency and coordinate with the emergency responders to ensure the user 16 receives the best possible medical care for their medical emergency.

A second transceiver 78 is integrated into the wrist watch 12 and the second transceiver 78 is electrically coupled to the control circuit 60. The second transceiver 78 is in wireless communication with a global positioning system 80 thereby facilitating the second transceiver 78 to establish a physical location of the wrist watch 12. The first transceiver 74 broadcasts the physical location of the wrist watch 12 to the emergency responders when the control circuit 60 receives the alert input to facilitate the emergency responders to locate the user 16. The second transceiver 78 may comprise a radio frequency transceiver or the like.

A plurality of biometric sensors 82 is provided and each of the biometric sensors 82 is integrated into the wrist watch 12 thereby facilitating each of the biometric sensors 82 to be in communication with the user's body 84. In this way each of the biometric sensors 82 can sense a respective one of a plurality of biometric parameters of the user's body 84. Each of the biometric sensors 82 is in communication with the communication unit 58 and the communication unit 58 communicates a distress alert to the emergency responders when any of the plurality of biometric sensors 82 senses that any of the respective biometric parameters of the user's body 84 is outside of a pre-determined range. In this way the plurality of biometric sensors 82 can facilitate the user 16 to request emergency assistance. Each of the biometric sensors 82 is integrated into the bottom wall 20 of the wrist watch 12 thereby facilitating the plurality of biometric sensors 82 to be in physical contact with the user 16's skin when the wrist watch 12 is worn. Additionally, each of the biometric sensors 82 is electrically coupled to the control circuit 60.

The plurality of biometric sensors 82 includes an accelerometer 86. The control circuit 60 receives the alert input when the accelerometer 86 detects deceleration which has an intensity exceeding a pre-determined intensity stored in the electronic memory 64. In this way the accelerometer 86 can detect when the user 16 has fallen. The plurality of biometric sensors 82 includes a glucose monitor 88 which measures the glucose level in the user 16's perspiration. The control circuit 60 receives the alert input when the glucose monitor 88 senses a glucose level that is above or below a pre-determined glucose threshold stored in the electronic memory 64. Additionally, the glucose monitor 88 may comprise an electronic glucose monitor specifically calibrated to measure glucose that is present in perspiration or the like.

The plurality of biometric sensors 82 includes a blood oxygen monitor 90 for measuring the oxygen level of the user 16's blood. The control circuit 60 receives the alert input when the blood oxygen monitor 90 measures an oxygen level that is below a pre-determined oxygen threshold stored in the electronic memory 64. The blood oxygen monitor 90 may comprise an infra-red blood oxygen monitor or other similar electronic monitor that can measure blood oxygen levels in a non-invasive manner.

The plurality of biometric sensors 82 includes a heart rate monitor 92 for measuring the user's 16 heart rate. The control circuit 60 receives the alert input when the heart rate monitor 92 measures a heart rate that is above or below a pre-determined pulse threshold stored in the electronic memory 64. The heart rate monitor 92 may comprise an optical heart rate monitor or other similar type of heart rate monitor that is commonly worn on a person's wrist for tracking heart rate. The pre-determined glucose threshold, the pre-determined oxygen threshold and the pre-determined pulse threshold may be determined by a physician and may be uploaded into the electronic memory 64 prior to being employed by the user 16.

A speaker 94 is integrated into the top wall 18 of the wrist watch 12 to emit audible sounds outwardly from the wrist watch 12 and the speaker 94 is electrically coupled to the control circuit 60. The audible sounds may include audible alerts, audible reminders or other pre-recorded sounds that are stored in the electronic memory 64. A microphone 95 is integrated the wrist watch 12 for detecting audible sounds and the microphone 95 is electrically coupled to the control circuit 60. The control circuit 60 receives a sound input when the microphone 95 detects audible sound that is above a pre-determined trigger intensity. The first transceiver 74 broadcasts the audible sound that the microphone detects to the to the user's 16 emergency contacts when the control circuit 60 receives the sound input. In this way user's 16 emergency contacts can be immediately notified that the user 16 is exposed to a risk such as an aggressive dog, a train, an oncoming vehicle or other loud noises that would indicate a potentially hazardous situation for the user 16.

A light emitter 96 is integrated into the top wall 18 of the wrist watch 12 to emit light outwardly from the wrist watch 12 and the light emitter 96 is electrically coupled to the control circuit 60. The light emitter 96 may comprise a light emitting diode or other type of electronic light emitter. A camera 98 is integrated into the top wall 18 of the wrist watch 12 to capture images and the camera 98 is electrically coupled to the control circuit 60. The camera 98 may comprise a digital video camera or other type of electronic camera commonly associated with smart devices, for example.

The control circuit 60 includes an electronic memory 99 which stores an interpretation program which translates American Sign Language into text. In this way the camera 98 can record sign language gestures made by the user 16 which are subsequently translated by the interpretation program into text that can be transmitted by the first transceiver 74 to the user's 16 emergency contacts or any other contact stored in the user's 16 personal electronic device 77. In this way the user 16 is facilitated to employ American Sign Language to communicate with their family members via the user's personal electronic device 77 when the first transceiver 74 is synchronized with the user's personal electronic device 77. Additionally, the first transceiver 74 may employ Bluetooth communication protocols to facilitate the first transceiver 74 to be synchronized with the user's 16 personal electronic device 77.

A plurality of control buttons 100 is each movably integrated into a respective one of the plurality of intersecting sides 24 of the perimeter wall 22 of the wrist watch 12 and each of the control buttons 100 is electrically coupled to the control circuit 60. Each of the control buttons 100 controls respective operational parameters of the control circuit 60, including but not being limited to, turning the light emitter 96 on and off, turning the camera 98 on and off and turning the speaker 94 on and off. A power supply 102 is integrated into the wrist watch 12 and the power supply 102 is electrically coupled to the control circuit 60. The power supply 102 comprises a rechargeable battery 104 that is integrated into the wrist watch 12 and the rechargeable battery 104 is electrically coupled to the control circuit 60. The power supply 102 includes a solar panel 106 is attached to the top wall 18 of the wrist watch 12 such that the solar panel 106 is exposed to sunlight. Additionally, the solar panel 106 is electrically coupled to the rechargeable battery 104 for charging the rechargeable battery 104.

A wrist band 108 is wearable around the user's wrist 14 and the wrist band 108 is releasably attachable to the pair of engagement elements 16 in the wrist watch 12. The wrist band 108 includes a first half 110 and a second half 112 and the wrist band 108 includes a pair of clasps 114. Each of the clasps 114 is attached to a first end 116 of a respective one of the first half 110 and the second half 112. Additionally, each of the clasps 114 has a pair of horns 118 each extending away from a respective first lateral side 120 and a second lateral side 122 of the clasps 114. Each of the clasps 114 has a plurality of holes 124 that is each integrated into the clasps 114 and each of the clasps 114 is slidable into a respective one of the slots 26 in the bottom wall 20 of the wrist watch 12.

Each of the clasps 114 is comprised of ferromagnetic material and the magnet 36 associated with a respective one of the sliding engagements 30 extends through a respective one of the holes 124 in a respective one of the clasps 114 when the respective clasp 114 is slid into the respective slot 26. In this way each of the first half 110 and the second half 112 is releasably attachable to the wrist watch 12. The first half 110 has a plurality of holes 126 each extending through a top surface 128 and a bottom surface 130 of the first half 110. The holes 126 in the first half 110 are spaced apart from each other and are distributed from a second end 131 of the first half 110 toward a first end of the first half 110.

The second half 112 has an opening 132 extending through a top surface 134 and a bottom surface 136 of the second half 112. The opening 132 is positioned closer to a second end 138 of the second half 112 than the first end 116 of the second half 112. Additionally, the second half 112 has a knob 140 that is coupled to and extends upwardly from the top surface 134 of the second half 112. The knob 140 on the second half 112 extends through a respective one of the holes 124 in the first half 110 such that the wrist band 108 forms a closed loop around the user's wrist 14.

In use, the wrist band 108 is worn around the user's wrist 14 such that the bottom wall 20 of the wrist watch 12 rests against the user's 16 skin. In this way each of the biometric sensors 82 is in contact with the skin on the user's wrist 14. Thus, the biometric sensors 82 can monitor each of the respective biometric parameters of the user's body 84. The communication unit 58 contacts the emergency responders and the user's 16 emergency contacts anytime one of the biometric sensors 82 senses a biometric parameter that is outside of the pre-determined range. In this way the emergency responders can immediately render medical aid to the user 16 without requiring the user 16 to manually call for help. Furthermore, the emergency responders can communicate with the user's 16 emergency contacts to ensure the emergency responders are given as much information as possible regarding the user 16. Additionally, the communication unit 58 contacts the emergency responders and the user's 16 emergency contacts when the accelerometer 86 determines that the user 16 has fallen down. In this way the user 16 can receive medical attention for almost any conceivable occurrence even if the user 16 is unable to call for help themselves. The camera 98 records American Sign Language gestures made by the user 16 to facilitate the hand gestures to be translated into text that is transmitted to anyone the user 16 wishes to communicate with via the user's personal electronic device 77.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A health monitoring assembly for monitoring the health of a physically limited user, said assembly comprising:
a wrist watch being positionable on a user's wrist, said wrist watch having a pair of engagement elements each being integrated into said wrist watch;
a communication unit being integrated into said wrist watch, said communication unit being in remote communication with emergency responders;
a plurality of biometric sensors, each of said biometric sensors being integrated into said wrist watch thereby facilitating each of said biometric sensors to be in communication with the user's body wherein each of said biometric sensors is configured to sense a respective one of a plurality of biometric parameters of the user's body, each of said biometric sensors being in communication with said communication unit, said communication unit communicating a distress alert to the emergency responders when any of said plurality of biometric sensors senses that any of the respective biometric parameters of the user's body is outside of a pre-determined range wherein said plurality of biometric sensors is configured to facilitate the user to request emergency assistance; and
a wrist band being wearable around a user's wrist, said wrist band being releasably attachable to said pair of engagement elements in said wrist watch.

2. The assembly according to claim 1, wherein said wrist watch has a top wall, a bottom wall and a perimeter wall extending between said top wall and said bottom wall, said perimeter wall having a plurality of intersecting sides such that said wrist watch has a decagonal shape, said wrist watch having a pair of slots each extending into said bottom wall such that each of said slots is associated with a respective one of said engagement elements, each of said slots extending laterally across a substantial width of said bottom wall, each of said slots intersecting a respective one of said plurality of intersecting sides of said perimeter wall, each of said slots being oriented parallel with each other, said pair of slots being spaced apart from a center of said bottom wall.

3. The assembly according to claim 2, wherein:
said assembly includes a pair of sliding engagements, each of said sliding engagements being slidably integrated within said wrist watch such that each of said sliding engagements is associated with a respective one of said engagement elements;
each of said sliding engagements includes a first member, a second member oriented at an angle with said first member and a magnet being disposed on a distal end of said second member with respect to said first member;
said first member has a front end, said front end being angled such that said front end slopes rearwardly between a top surface of said first member and a bottom surface of said first member, said second member being disposed on said bottom surface, said magnet being oriented to extend toward said front end of said first member;
each of said sliding engagements includes a slide comprising a stem extending upwardly from said top surface of said first member and a head being transversely oriented with said stem, said head slidably engaging said top wall of said wrist watch;
each of said sliding engagements is aligned with a respective one of said slots in said bottom wall of said wrist watch;
each of said sliding engagements is biased into a first position having said magnet extending across said respective slot, each of said sliding engagements being urgeable into a second position having said magnet being displaced from said respective slot.

4. The assembly according to claim 3, further comprising a pair of release buttons, each of said release buttons being movably integrated into said bottom wall of said wrist watch, each of said release buttons including a knob extending into an interior of said wrist watch, said knob associated with each of said release buttons being aligned with said front end of said first member of a respective one of said sliding engagements, said knob of a respective one of said release buttons engaging said front end of said first member of said respective sliding engagement when said respective release button is depressed thereby urging said respective sliding engagement into said second position.

5. The assembly according to claim 1, wherein said communication unit comprises:
a control circuit being integrated into said wrist watch, said control circuit receiving an alert input, said control circuit including an electronic timer for tracking the time of day and the calendar date, said control circuit including an electronic memory;
a touch screen being integrated into said wrist watch thereby facilitating said touch screen to be touched by a user, said touch screen being electrically coupled to said control circuit, said touch screen displaying time indicia comprising numbers to indicate a time of day, said touch screen displaying date indicia comprising words and numbers to indicate the calendar date, said touch screen displaying menu indicia comprising an graphical user interface thereby facilitating the user to navigate functional parameters of said control circuit;
a first transceiver being integrated into said wrist watch, said first transceiver being electrically coupled to said control circuit, said first transceiver being in wireless communication with an extrinsic communication network thereby facilitating said first transceiver to be in remote communication with the emergency responders, said first transceiver being actuated to broadcast a distress alert to the emergency responders when said control circuit receives said alert input; and
a second transceiver being integrated into said wrist watch, said second transceiver being electrically coupled to said control circuit, said second transceiver being in wireless communication with a global positioning system thereby facilitating said second transceiver to establish a physical location of said wrist watch, said first transceiver broadcasting the physical location of said wrist watch to the emergency responders when said control circuit receives said alert input wherein said second transceiver is configured to facilitate the emergency responders to locate the user.

6. The assembly according to claim 5, wherein each of said biometric sensors is integrated into said bottom wall of said wrist watch thereby facilitating said plurality of biometric sensors to be in physical contact with the user's skin when said wrist watch is worn, each of said biometric sensors being electrically coupled to said control circuit.

7. The assembly according to claim 6, wherein said plurality of biometric sensors includes:
an accelerometer, said control circuit receiving said alert input when said accelerometer detects deceleration having an intensity exceeding a pre-determined intensity stored in said electronic memory wherein said accelerometer is configured to detect when the user has fallen;
a glucose monitor measuring the glucose level in the user's perspiration, said control circuit receiving said alert input when said glucose monitor senses a glucose level that is above or below a pre-determined glucose threshold stored in said electronic memory;
a blood oxygen monitor measuring the oxygen level of the user's blood, said control circuit receiving said alert input when said blood oxygen monitor measures an oxygen level that is below a pre-determined oxygen threshold stored in said electronic memory; and
a heart rate monitor measuring the user's heart rate, said control circuit receiving said alert input when said heart rate monitor measures a heart rate that is above or below a pre-determined pulse threshold stored in said electronic memory.

8. The assembly according to claim 5, further comprising:
a speaker being integrated into said wrist watch wherein said speaker is configured to emit audible sounds outwardly from said wrist watch, said speaker being electrically coupled to said control circuit;
a light emitter being integrated into said wrist watch wherein said light emitter is configured to emit light outwardly from said wrist watch, said light emitter being electrically coupled to said control circuit; and
a camera being integrated into said wrist watch wherein said camera is configured to capture images, said camera being electrically coupled to said control circuit.

9. The assembly according to claim 5, further comprising a plurality of control buttons, each of said control buttons being movably integrated into said wrist watch, each of said control buttons being electrically coupled to said control circuit, each of said control buttons controlling respective operational parameters of said control circuit.

10. The assembly according to claim 5, further comprising a power supply being integrated into said wrist watch, said power supply being electrically coupled to said control circuit, said power supply comprising:
a rechargeable battery being integrated into said wrist watch, said rechargeable battery being electrically coupled to said control circuit; and
a solar panel being attached to said wrist watch wherein said solar panel is configured to be exposed to sunlight, said solar panel being electrically coupled to said rechargeable battery for charging said rechargeable battery.

11. The assembly according to claim 3, wherein:
said wrist band includes a first half and a second half, said wrist band including a pair of clasps, each of said clasps being attached to a first end of a respective one of said first half and said second half, each of said clasps having a pair of horns each extending away from a respective first lateral side and a second lateral side of said clasps, each of said claps having a plurality of holes being integrated into said clasps;
each of said clasps is slidable into a respective one of said slots in said bottom wall of said wrist watch, each of said clasps being comprised of ferromagnetic material;
said magnet associated with a respective one of said sliding engagements extends through a respective one of said holes in a respective one of said clasps when said respective clasp is slid into said respective slot for releasably attaching each of said first half and said second half to said wrist watch.

12. The assembly according to claim 11, wherein:
said first half has a plurality of holes each extending through a top surface and a bottom surface of said first half, said holes being spaced apart from each other and being distributed from said second end of said first half toward a first end of said first half;
said second half has an opening extending through a top surface and a bottom surface of said second half, said opening being positioned closer to a second end of said second half than a first end of said second half; and
said second half has a knob being coupled to and extending upwardly from said top surface of said second half, said knob extending through a respective one of said holes in said first half such that said wrist band forms a closed loop around the user's wrist.

13. A health monitoring assembly for monitoring the health of a physically limited user, said assembly comprising:
a wrist watch being positionable on a user's wrist, said wrist watch having a pair of engagement elements each being integrated into said wrist watch, said wrist watch having a top wall, a bottom wall and a perimeter wall extending between said top wall and said bottom wall, said perimeter wall having a plurality of intersecting sides such that said wrist watch has a decagonal shape, said wrist watch having a pair of slots each extending into said bottom wall such that each of said slots is associated with a respective one of said engagement elements, each of said slots extending laterally across a substantial width of said bottom wall, each of said slots intersecting a respective one of said plurality of intersecting sides of said perimeter wall, each of said slots being oriented parallel with each other, said pair of slots being spaced apart from a center of said bottom wall;
a pair of sliding engagements, each of said sliding engagements being slidably integrated within said wrist watch such that each of said sliding engagements is associated with a respective one of said engagement elements, each of said sliding engagements including a first member, a second member oriented at an angle with said first member and a magnet being disposed on a distal end of said second member with respect to said first member, said first member having a front end, said front end being angled such that said front end slopes rearwardly between a top surface of said first member and a bottom surface of said first member, said second member being disposed on said bottom surface, said magnet being oriented to extend toward said front end of said first member, each of said sliding engagements includes a slide comprising a stem extending upwardly from said top surface of said first member and a head being transversely oriented with said stem, said head slidably engaging said top wall of said wrist watch, each of said sliding engagements being aligned with a respective one of said slots in said bottom wall of said wrist watch, each of said sliding engagements being biased into a first position having said magnet extending across said respective slot, each of said sliding engagements being urgeable into a second position having said magnet being displaced from said respective slot;
a pair of release buttons, each of said release buttons being movably integrated into said bottom wall of said wrist watch, each of said release buttons including a knob extending into an interior of said wrist watch, said knob associated with each of said release buttons being aligned with said front end of said first member of a respective one of said sliding engagements, said knob of a respective one of said release buttons engaging said front end of said first member of said respective sliding engagement when said respective release button is depressed thereby urging said respective sliding engagement into said second position;
a communication unit being integrated into said wrist watch, said communication unit being in remote communication with emergency responders, said communication unit comprising:
a control circuit being integrated into said wrist watch, said control circuit receiving an alert input, said control circuit including an electronic timer for tracking the time of day and the calendar date, said control circuit including an electronic memory;
a touch screen being integrated into said top wall of said wrist watch thereby facilitating said touch screen to be touched by a user, said touch screen being electrically coupled to said control circuit, said touch screen displaying time indicia comprising numbers to indicate a time of day, said touch screen displaying date indicia comprising words and numbers to indicate the calendar date, said touch screen displaying menu indicia comprising an graphical user interface thereby facilitating the user to navigate functional parameters of said control circuit;
a first transceiver being integrated into said wrist watch, said first transceiver being electrically coupled to said control circuit, said first transceiver being in wireless communication with an extrinsic communication network thereby facilitating said first transceiver to be in remote communication with the emergency responders, said first transceiver being actuated to broadcast a distress alert to the emergency responders when said control circuit receives said alert input; and
a second transceiver being integrated into said wrist watch, said second transceiver being electrically coupled to said control circuit, said second transceiver being in wireless communication with a global positioning system thereby facilitating said second transceiver to establish a physical location of said wrist watch, said first transceiver broadcasting the physical location of said wrist watch to the emergency responders when said control circuit receives said alert input wherein said second transceiver is configured to facilitate the emergency responders to locate the user;
a plurality of biometric sensors, each of said biometric sensors being integrated into said wrist watch thereby facilitating each of said biometric sensors to be in communication with the user's body wherein each of said biometric sensors is configured to sense a respective one of a plurality of biometric parameters of the user's body, each of said biometric sensors being in communication with said communication unit, said communication unit communicating a distress alert to the emergency responders when any of said plurality of biometric sensors senses that any of the respective biometric parameters of the user's body is outside of a pre-determined range wherein said plurality of biometric sensors is configured to facilitate the user to request emergency assistance, each of said biometric sensors being integrated into said bottom wall of said wrist watch thereby facilitating said plurality of biometric sensors to be in physical contact with the user's skin when said wrist watch is worn, each of said biometric sensors being electrically coupled to said control circuit, said plurality of biometric sensors including:
an accelerometer, said control circuit receiving said alert input when said accelerometer detects deceleration having an intensity exceeding a pre-determined intensity stored in said electronic memory wherein said accelerometer is configured to detect when the user has fallen;
a glucose monitor measuring the glucose level in the user's perspiration, said control circuit receiving said alert input when said glucose monitor senses a glucose level that is above or below a pre-determined glucose threshold stored in said electronic memory;
a blood oxygen monitor measuring the oxygen level of the user's blood, said control circuit receiving said alert input when said blood oxygen monitor measures an oxygen level that is below a pre-determined oxygen threshold stored in said electronic memory; and
a heart rate monitor measuring the user's heart rate, said control circuit receiving said alert input when said heart rate monitor measures a heart rate that is above or below a pre-determined pulse threshold stored in said electronic memory;
a speaker being integrated into said top wall of said wrist watch wherein said speaker is configured to emit audible sounds outwardly from said wrist watch, said speaker being electrically coupled to said control circuit;
a light emitter being integrated into said top wall of said wrist watch wherein said light emitter is configured to emit light outwardly from said wrist watch, said light emitter being electrically coupled to said control circuit;
a camera being integrated into said top wall of said wrist watch wherein said camera is configured to capture images, said camera being electrically coupled to said control circuit;
a plurality of control buttons, each of said control buttons being movably integrated into a respective one of said plurality of intersecting sides of said perimeter wall of said wrist watch, each of said control buttons being electrically coupled to said control circuit, each of said control buttons controlling respective operational parameters of said control circuit;
a power supply being integrated into said wrist watch, said power supply being electrically coupled to said control circuit, said power supply comprising:
a rechargeable battery being integrated into said wrist watch, said rechargeable battery being electrically coupled to said control circuit; and
a solar panel being attached to said top wall of said wrist watch wherein said solar panel is configured to be exposed to sunlight, said solar panel being electrically coupled to said rechargeable battery for charging said rechargeable battery; and
a wrist band being wearable around a user's wrist, said wrist band being releasably attachable to said pair of engagement elements in said wrist watch, said wrist band including a first half and a second half, said wrist band including a pair of clasps, each of said clasps being attached to a first end of a respective one of said first half and said second half, each of said clasps having a pair of horns each extending away from a respective first lateral side and a second lateral side of said clasps, each of said claps having a plurality of holes being integrated into said clasps, each of said clasps being slidable into a respective one of said slots in said bottom wall of said wrist watch, each of said clasps being comprised of ferromagnetic material, said magnet associated with a respective one of said sliding engagements extending through a respective one of said holes in a respective one of said clasps when said respective clasp is slid into said respective slot for releasably attaching each of said first half and said second half to said wrist watch, said first half having a plurality of holes each extending through a top surface and a bottom surface of said first half, said holes being spaced apart from each other and being distributed from said second end of said first half toward a first end of said first half, said second half having an opening extending through a top surface and a bottom surface of said second half, said opening being positioned closer to a second end of said second half than a first end of said second half, said second half having a knob being coupled to and extending upwardly from said top surface of said second half, said knob extending through a respective one of said holes in said first half such that said wrist band forms a closed loop around the user's wrist.
